(19) United States
(12) Patent Application Publication
(10) Pub. No.: US 2022/0249520 A1
(43) Pub. Date: Aug. 11, 2022

(54) COMPOSITION FOR TREATING OR PREVENTING MILD COGNITIVE IMPAIRMENT

(71) Applicants: Kenichi KOZU, Tokyo (JP); HBC Funato Co., Ltd., Tokyo (JP)

(72) Inventor: Kenichi KOZU, Tokyo (JP)

(73) Assignees: HBC Funato Co., Ltd., Tokyo (JP); Kenichi Kozu, Tokyo (JP)

(21) Appl. No.: 17/174,042

(22) Filed: Feb. 11, 2021

(51) Int. Cl. A61K 31/661; A61K 47/36; A61K 47/42; A61K 9/48; A61P 25/28; A61K 47/44

(52) U.S. Cl. CPC A61K 31/661; A61K 47/36; A61K 47/42; A61K 47/44; A61K 9/4825; A61K 9/4808; A61P 25/28; A61K 9/4858

(57) ABSTRACT

A composition for treating or preventing mild cognitive impairment is provided. The composition for treating or preventing mild cognitive impairment includes a compound having a structure represented by the formula I and an auxiliary component. In the formula I, R₁ and R₂ are each independently hydrogen or —COR₃ with the proviso that R₁ and R₂ are not —COR₃ at the same time, R₃ is a saturated or unsaturated, C₁₋₃₀ aliphatic hydrocarbon group, and A is —CH₂—CH₂—N⁺(CH₃)₃, —CH₂—CH₂—N⁺H₃, —CH₂—CH(NH₂)(COO⁻), or a C₃₋₆ cycloalkyl optionally substituted with 1 to 6 substituents selected from the group consisting of C₁₋₆ alkyls, C₁₋₆ alkoxies and hydroxyl groups. The composition is administered to a subject identified as having mild cognitive impairment with a brain wave analysis system in a dose of 8 to 60 mg/kg body weight per day in terms of the compound having a structure of the formula I.

### BACKGROUND OF THE INVENTION

### Field of the Invention

The present invention relates to compositions for treating or preventing mild cognitive impairment.

### Description of the Related Art

Dementia is a central nervous system disease in which brain functions such as cognitive functions decline, and is seen in an increasing number of patients in Japan. Advanced dementia involves symptoms such as profound forgetfulness and wandering, which can increase the care burden. These facts have made the disease a major social problem in Japan that is experiencing a super-aging society.

Dementia is usually treated symptomatically with compositions including such components as cholinesterase inhibitors and NMDA receptor antagonists. However, the treatment generally starts only after patients are diagnosed as having dementia by doctors. Radical treatment which restores the cognitive functions such as memory ability of the patients has not been established yet.

In recent years, mild cognitive impairment (hereinafter, sometimes abbreviated as “MCI”) is being recognized as a prodromal stage of dementia. It is known that mild cognitive impairment is not as severe as dementia but does involve impairments in cognitive and other functions without interfering with daily life. According to a report, individuals with mild cognitive impairment develop dementia with a probability of about 10% in 1 year and with a probability of about 40% in 5 years (Non Patent Literature 1). On the other hand, studies suggest that, unlike dementia, patients with mild cognitive impairment could be radically treated and could recover cognitive functions such as memory ability back to the healthy conditions. Thus, it is expected that if a person who has or potentially has mild cognitive impairment can be detected and identified at an early stage, and be given a composition effective for treating or preventing mild cognitive impairment, the onset of dementia itself can be prevented.

### CITATION LIST

### Non Patent Literature

Non Patent Literature 1: Maddalena Bruscoli and Simon Lovestone, International Psychogeriatrics, 2004, 16:2, 129-140.

### SUMMARY OF THE INVENTION

Unfortunately, at present, no methods have been established that are capable of objectively identifying whether or not a person has mild cognitive impairment, let alone no compositions for treating a person identified as having mild cognitive impairment or for preventing a potential patient from the onset of the disease.

The present invention provides a composition for treating or preventing mild cognitive impairment.

In light of the problems discussed above, the present inventor carried out extensive studies and has found that, surprisingly, a subject identified as having mild cognitive impairment with a brain wave analysis system can be treated or prevented from the onset or progress of the disease by the administration of a predetermined dose of a composition that includes a compound having a structure of the formula I and an auxiliary component. The present invention has been completed based on the finding.

Specifically, the present invention in summary provides the following.

[1] A composition for treating or preventing mild cognitive impairment, including a compound having a structure represented by the formula I below and an auxiliary component,

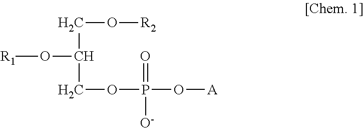

wherein R₁ and R₂ are each independently hydrogen or —COR₃ with the proviso that R₁ and R₂ are not —COR₃ at the same time,

R₃ is a saturated or unsaturated, C₁₋₃₀ aliphatic hydrocarbon group, and

A is —CH₂—CH₂—N⁺(CH₃)₃, —CH₂—CH₂—N⁺H₃, —CH₂—CH(NH₂)(COO⁻), or a C₃₋₆ cycloalkyl optionally substituted with 1 to 6 substituents selected from the group consisting of C₁₋₆ alkyls, C₁₋₆ alkoxies and hydroxyl groups,

the composition being administered to a subject identified as having mild cognitive impairment with a brain wave analysis system in a dose of 8 to 60 mg/kg body weight per day in terms of the compound having a structure of the formula I.

[2] The composition described in [1], wherein R₁ is hydrogen and R₂ is —COR₃.

[3] The composition described in [1] or [2], wherein A is —CH₂—CH₂—N⁺(CH₃)₃.

[4] The composition described in any one of [1] to [3], wherein the compound having a structure of the formula I is a lysolecithin.

[5] The composition described in [4], wherein the lysolecithin is soybean lysolecithin.

[6] The composition described in any one of [1] to [5], wherein the subject identified as having mild cognitive impairment with a brain wave analysis system is a subject having a similarity value with respect to general AD of more than 0 as measured with brain wave analysis system NATESAS.

[7] The composition described in any one of [1] to [6], which includes the auxiliary component in an amount of 1 to 16 parts by mass per part by mass of the compound having a structure of the formula I.

[8] The composition described in any one of [1] to [7], wherein the auxiliary component includes at least one selected from the group consisting of nucleic acids, collagens, vitamin B1, vitamin B2, vitamin B6, vitamin B12, vitamin C, niacin, pantothenic acid, lactic acid bacteria-producing substances, docosahexaenoic acid and eicosapentaenoic acid.

[1A] A method for treating or preventing mild cognitive impairment in a subject identified as having mild cognitive impairment with a brain wave analysis system, the method including:

a step of bringing brain waves of the subject to or close to brain waves of a healthy person by administering to the subject a composition including a compound having a structure represented by the formula I below and an auxiliary component,

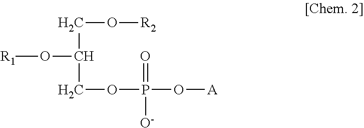

wherein R₁ and R₂ are each independently hydrogen or —COR₃ with the proviso that R₁ and R₂ are not —COR₃ at the same time,

R₃ is a saturated or unsaturated, C₁₋₃₀ aliphatic hydrocarbon group, and

A is —CH₂—CH₂—N⁺(CH₃)₃, —CH₂—CH₂—N⁺H₃, —CH₂—CH(NH₂)(COO⁻), or a C₃₋₆ cycloalkyl optionally substituted with 1 to 6 substituents selected from the group consisting of C₁-6 alkyls, C₁₋₆ alkoxies and hydroxyl groups,

the dose of the composition administered to the subject in the step being 8 to 60 mg/kg body weight per day in terms of the compound having a structure of the formula I.

[2A] The method described in [1A], wherein in the compound having a structure of the formula I, R₁ is hydrogen and R₂ is —COR₃.

[3A] The method described in [1A], wherein in the compound having a structure of the formula I, A is —CH₂—CH₂—N⁺(CH₃)₃.

[4A] The method described in [1A], wherein the compound having a structure of the formula I is a lysolecithin.

[5A] The method described in [4A], wherein the lysolecithin is soybean lysolecithin.

[6A] The method described in [1A], wherein the subject identified as having mild cognitive impairment with a brain wave analysis system is a subject having a similarity value with respect to general AD of more than 0 as measured with brain wave analysis system NATESAS.

[7A] The method described in [1A], wherein the composition includes the auxiliary component in an amount of 1 to 16 parts by mass per part by mass of the compound having a structure of the formula I.

[8A] The method described in [1A], wherein the auxiliary component includes at least one selected from the group consisting of nucleic acids, collagens, vitamin B1, vitamin B2, vitamin B6, vitamin B12, vitamin C, niacin, pantothenic acid, lactic acid bacteria-producing substances, docosahexaenoic acid and eicosapentaenoic acid.

According to the present invention, it is possible to objectively identify a subject having mild cognitive impairment. The identified subject is given a composition including a compound with a structure of the formula I and an auxiliary component, in a predetermined manner and a predetermined dose so as to treat or prevent mild cognitive impairment. Further, the composition of the present invention can bring brain waves of the subject having mild cognitive impairment to or close to brain waves of a healthy person. Thus, the present invention allows for the radical treatment of dementia and the radical prevention of the onset of dementia which are far from possible with the conventional therapeutic agents, methods and other approaches.

Further, the composition according to the present invention includes not only the compound having a structure of the formula I but also an auxiliary component. While the compound having a structure of the formula I alone is negligibly or insufficiently effective in treating or preventing mild cognitive impairment, the composition attains a marked enhancement in the therapeutic or preventive effects on mild cognitive impairment. The compound with a structure of the formula I can be an essential nutrient to brain cells, and the auxiliary component can be an additional nutrient. They in combination form a complex nutrient that presumably enhances the metabolism of brain cells and/or brain tissues, and consequently dementia symptoms are improved.

As described above, the present invention makes it possible to radically treat or prevent dementia which has become a social problem in Japan, and thus can arrest the increase in care burden and the increase in medical expenses stemming from the super-aging of the society, thus contributing to the medical economy and the like.

### BRIEF DESCRIPTION OF THE DRAWINGS

FIG. 1 illustrates color maps of brain waves (states of brain activity) of a healthy person, general AD, CL1, CL2, CL3 and CL4 constructed in brain wave analysis system NATESAS;

FIG. 2A illustrates color maps showing the results of the analysis of brain waves (states of brain activity) of Subject 1 in Test Example 1 before, 40 minutes after and 1 month after the ingestion of a composition of Example 1, with use of brain wave analysis system NATESAS;

FIG. 2B illustrates variations in membership probability in Subject 1 of Test Example 1 before and 1 month after the ingestion of a composition of Example 1;

FIG. 3A illustrates color maps showing the results of the analysis of brain waves (states of brain activity) of Subject 2 in Test Example 1 before, 40 minutes after and 1 month after the ingestion of a composition of Example 1, with use of brain wave analysis system NATESAS;

FIG. 3B illustrates variations in membership probability in Subject 2 of Test Example 1 before and 1 month after the ingestion of a composition of Example 1;

FIG. 4A illustrates color maps showing the results of the analysis of brain waves (states of brain activity) of Subject 3 in Test Example 1 before, 40 minutes after and 1 month after the ingestion of a composition of Example 1, with use of brain wave analysis system NATESAS; and

FIG. 4B illustrates variations in membership probability in Subject 3 of Test Example 1 before and 1 month after the ingestion of a composition of Example 1.

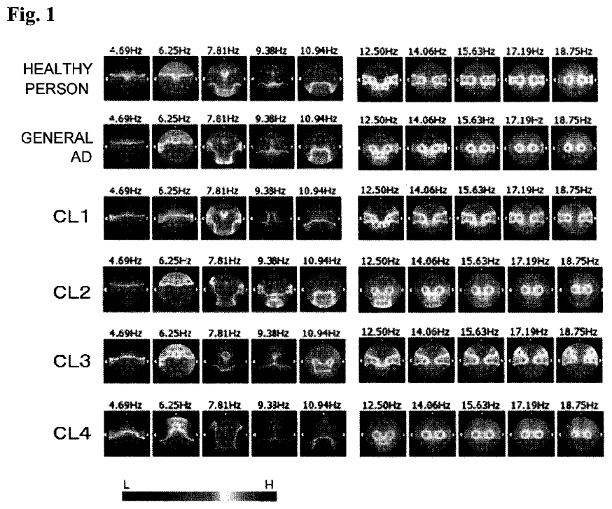

### DETAILED DESCRIPTION OF THE INVENTION

In an embodiment of the present invention, a composition for treating or preventing mild cognitive impairment is provided which includes a compound having a structure represented by the formula I below and an auxiliary component.

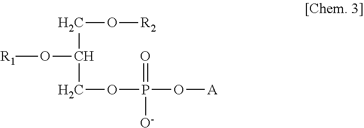

In the formula, R₁ and R₂ are each independently hydrogen or —COR₃ with the proviso that R₁ and R₂ are not —COR₃ at the same time,

R₃ is a saturated or unsaturated, C₁₋₃₀ aliphatic hydrocarbon group, and

A is —CH₂—CH₂—N⁺(CH₃)₃, —CH₂—CH₂—N⁻H₃, —CH₂—CH(NH₂)(COO⁻), or a C₃₋₆ cycloalkyl optionally substituted with 1 to 6 substituents selected from the group consisting of C₁₋₆ alkyls, C₁₋₆ alkoxies and hydroxyl groups. The composition is administered to a subject identified as having mild cognitive impairment with a brain wave analysis system in a dose of 8 to 60 mg/kg body weight per day in terms of the compound having a structure of the formula I.

In another embodiment of the present invention, a method for treating or preventing mild cognitive impairment is provided which includes administering, to a subject identified as having mild cognitive impairment with a brain wave analysis system, a composition that includes a compound having a structure represented by the formula I and an auxiliary component in a dose of 8 to 60 mg/kg body weight per day in terms of the compound having a structure of the formula I.

In another embodiment of the present invention, use is provided of a compound having a structure of the formula I in the production of a composition for treating or preventing mild cognitive impairment that includes the compound and an auxiliary component, the use including administering the composition to a subject identified as having mild cognitive impairment with a brain wave analysis system in a dose of 8 to 60 mg/kg body weight per day in terms of the compound having a structure of the formula I.

As used herein, the “saturated or unsaturated, C₁₋₃₀ aliphatic hydrocarbon group” means a saturated or unsaturated, linear or branched hydrocarbon group containing 1 to 30 carbon atoms (with examples including, but not limited to, methyl, ethyl, propyl, isopropyl, n-butyl, i-butyl, t-butyl, n-pentyl, n-hexyl, n-heptyl, n-octyl, n-nonyl, n-decyl, undecyl, dodecyl, tridecyl, tetradecyl, pentadecyl, hexadecyl, heptadecyl, octadecyl, nonadecyl, eicosyl, heneicosyl, docosyl, tricosyl, tetracosyl, pentacosyl, hexacosyl, heptacosyl, octacosyl, nonacosyl, triacontyl, CH₃—(CH₂)₅CH═CH(CH₂)₇—, CH₃—(CH₂)₇CH═CH(CH₂)₇—, CH₃—(CH₂)₅CH═CH(CH₂)₉—, CH₃—(CH₂)₃(CH₂CH═CH)₂(CH₂)₇—, CH₃—(CH₂CH═CH)₃(CH₂)₇—, CH₃—(CH₂)₃(CH₂CH═CH)₃(CH₂)₄—, CH₃—(CH₂)₃(CH═CH)₃(CH₂)₇—, CH₃—(CH₂)₆(CH₂CH═CH)₂(CH₂)₆—, CH₃—(CH₂)₆(CH₂CH═CH)₃(CH₂)₃—, CH₃—(CH₂)₃(CH₂CH═CH)₄(CH₂)₃—, CH₃—(CH₂)₇CH₂CH═CH(CH₂)₁₃—, CH₃—CH₂(CH═CHCH₂)₆CH₂—, and CH₃—CH₂(CH═CHCH₂)₅(CH₂)₂—). Preferred saturated or unsaturated, C₁₋₃₀ aliphatic hydrocarbon groups are tridecyl, tetradecyl, pentadecyl, hexadecyl, heptadecyl, octadecyl, CH₃—(CH₂)₇CH═CH(CH₂)₇—, CH₃—(CH₂)₃(CH₂CH═CH)₂(CH₂)₇—, CH₃—CH₂(CH═CHCH₂)₆CH₂—, and CH₃—CH₂(CH═CHCH₂)₅(CH₂)₂—.

As used herein, the “C₁₋₆ alkyl” means a saturated, linear or branched hydrocarbon group containing 1 to 6 carbon atoms (with examples including, but not limited to, methyl, ethyl, propyl, isopropyl, n-butyl, i-butyl, t-butyl, n-pentyl and n-hexyl). Preferred C₁-6 alkyls are C₁₋₄ alkyls (such as, for example, methyl, ethyl, propyl, butyl and isopropyl). Methyl and ethyl are more preferable.

As used herein, the “C₁₋₆ alkoxy” means a group having a structure of the formula “—O—C₁₋₆ alkyl” (with examples including, but not limited to, methoxy, ethoxy, propoxy, isopropoxy and butoxy). Preferred C₁-6 alkoxies are methoxy, ethoxy and propoxy. Methoxy and ethoxy are more preferable.

As used herein, the “hydroxyl group” means a group having an —OH structure.

As used herein, the “C₃₋₆ cycloalkyl” means a saturated, monocyclic or bicyclic hydrocarbon group containing 3 to 6 carbon atoms (with examples including, but not limited to, cyclopropyl, cyclobutyl and cyclohexyl). A preferred C₃₋₆ cycloalkyl is cyclohexyl.

As used herein, the “C₃-6 cycloalkyl optionally substituted with 1 to 6 substituents selected from the group consisting of C₁₋₆ alkyls, C₁-6 alkoxies and hydroxyl groups” means a C₃-6 cycloalkyl which may be or may not be substituted with 1 to 6 substituents selected from the group consisting of C₁₋₆ alkyls, C₁-6 alkoxies and hydroxyl groups. The C₃-6 cycloalkyl optionally substituted with 1 to 6 substituents selected from the group consisting of C₁₋₆ alkyls, C₁₋₆ alkoxies and hydroxyl groups is preferably cyclohexyl optionally substituted with 1 to 6 substituents selected from the group consisting of C₁₋₆ alkyls, C₁₋₆ alkoxies and hydroxyl groups, more preferably cyclohexyl substituted with 1 to 6 substituents selected from the group consisting of C₁-6 alkyls, C₁₋₆ alkoxies and hydroxyl groups, still more preferably cyclohexyl substituted with 1 to 6 hydroxyl groups, further preferably cyclohexyl substituted with 6 hydroxyl groups, and still further preferably inositol.

In an embodiment of the present invention, R₁ is hydrogen.

In an embodiment of the present invention, R₂ is —COR₃.

In an embodiment of the present invention, R₁ is hydrogen and R₂ is —COR₃.

In an embodiment of the present invention, A is —CH₂—CH₂—N⁺(CH₃)₃.

In an embodiment of the present invention, R₁ is hydrogen and A is —CH₂—CH₂—N⁺(CH₃)₃.

In an embodiment of the present invention, R₂ is —COR₃ and A is —CH₂—CH₂—N⁺(CH₃)₃.

In an embodiment of the present invention, R₁ is hydrogen, R₂ is —COR₃, and A is —CH₂—CH₂—N⁺(CH₃)₃.

In a preferred embodiment of the present invention, the compound having a structure of the formula I has a structure of the formula II below.

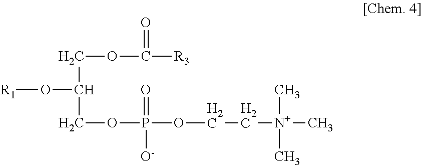

In a more preferred embodiment of the present invention, the compound represented by the formula I is a lysolecithin.

As used herein, the “lysolecithin” may be a lysolecithin of a single structure or may be a mixture of lysolecithins having different structures. Examples of the lysolecithins include, but are not limited to, soybean lysolecithin and egg yolk lysolecithin. The lysolecithins may be used singly, or two or more may be used in combination. In some cases, such lysolecithins as soybean lysolecithin and egg yolk lysolecithin are mixtures of different structures.

An embodiment of the present invention resides in a composition described in the present specification in which the lysolecithin is soybean lysolecithin.

As used herein, the “auxiliary component” may be any component without limitation which does not belong to the compounds having a structure of the formula I. Examples of the auxiliary components include, but are not limited to, excipients, lubricants, binders, disintegrants, pH regulators, solvents, solubilizers, suspending agents, emulsifiers, gelling agents, isotonic agents, buffers, soothing agents, preservatives, antioxidants, colorants, sweeteners, surfactants, other components useful for subjects, and other components generally used in formulation and other processes.

The auxiliary components may be used singly, or two or more may be used in combination.

Examples of the excipients include, but are not limited to, lactose hydrate, white sugar, glucose, starch, sucrose, crystalline cellulose, mannitol, starch syrup, reduced starch syrup, high-fructose corn syrup and maple syrup. The excipients may be used singly, or two or more may be used in combination.

Examples of the lubricants include, but are not limited to, light anhydrous silicic acid, stearic acid, magnesium stearate, calcium stearate, sucrose fatty acid esters, polyethylene glycol and talc. The lubricants may be used singly, or two or more may be used in combination.

Examples of the binders include, but are not limited to, gum arabic, crystalline cellulose, sucrose, mannitol, dextrin, hydroxypropylcellulose, hydroxymethylcellulose and polyvinylpyrrolidone. The binders may be used singly, or two or more may be used in combination.

Examples of the disintegrants include, but are not limited to, starch, carboxymethylcellulose, carboxymethylcellulose calcium, croscarmellose sodium, croscarmellose calcium, carboxymethyl starch sodium, crospovidone and low-substituted hydroxypropylcellulose. The disintegrants may be used singly, or two or more may be used in combination.

Examples of the pH regulators include, but are not limited to, acetic acid, lactic acid, tartaric acid, oxalic acid, glycolic acid, malic acid, citric acid, succinic acid, fumaric acid, phosphoric acid, hydrochloric acid, sulfuric acid, nitric acid, salts thereof, sodium hydroxide, potassium hydroxide, sodium hydrogencarbonate and potassium carbonate. The pH regulators may be used singly, or two or more may be used in combination.

Examples of the solvents include, but are not limited to, waters such as tap water, normal water, distilled water, purified water and water for injection; alcohols such as methanol, ethanol, propanol and isopropanol; acetone; single fatty acids such as acetic acid, propanoic acid, butanoic acid, pentanoic acid, hexanoic acid, heptanoic acid, myristic acid, stearic acid and oleic acid, and esters thereof; vegetable oils such as sesame oil, peanut oil, coconut oil, palm oil, soybean oil, olive oil, coconut oil, corn oil, cottonseed oil, castor oil, rapeseed oil, sunflower oil, safflower oil and linseed oil; propylene glycol; and macrogol. The solvents may be used singly, or two or more may be used in combination.

Examples of the solubilizers include, but are not limited to, polyethylene glycol; propylene glycol; cyclodextrin; sugar alcohols such as mannitol; benzyl benzoate; tris-aminomethane; cholesterol; triethanolamine; sodium carbonate; sodium citrate; alcohols such as methanol, ethanol, propanol and isopropanol; single fatty acids such as acetic acid, propanoic acid, butanoic acid, pentanoic acid, hexanoic acid, heptanoic acid, myristic acid, stearic acid and oleic acid, and esters thereof; and vegetable oils such as sesame oil, peanut oil, coconut oil, palm oil, soybean oil, olive oil, coconut oil, corn oil, cottonseed oil, castor oil, rapeseed oil and sunflower oil. The solubilizers may be used singly, or two or more may be used in combination.

Examples of the suspending agents include, but are not limited to, stearyltriethanolamine, sodium laurylsulfate, laurylaminopropionic acid, stearic acid monoglyceride, polyvinyl alcohol, polyvinylpyrrolidone, sodium carboxymethylcellulose, methylcellulose, hydroxymethylcellulose, hydroxyethylcellulose and hydroxypropylcellulose. The suspending agents may be used singly, or two or more may be used in combination.

Examples of the emulsifiers include, but are not limited to, glycerin fatty acid esters such as stearic acid monoglyceride and oleic acid monoglyceride; sorbitan fatty acid esters; oxyethylene fatty acid alcohols; sodium oleate; morpholine fatty acid salts; propylene glycol fatty acid esters; saponin; and casein sodium. The emulsifiers may be used singly, or two or more may be used in combination.

Examples of the gelling agents include, but are not limited to, carrageenan, gelatin, pectin, locust bean gum, starch (including modified starch), gellan gum, alginic acid and salts thereof, carboxymethylcellulose, hydroxyethylcellulose and hydroxypropylmethylcellulose. The gelling agents may be used singly, or two or more may be used in combination.

Examples of the isotonic agents include, but are not limited to, sodium chloride, glycerin and mannitol. The isotonic agents may be used singly, or two or more may be used in combination.

Examples of the buffers include, but are not limited to, phosphoric acid salts, acetic acid salts, carbonic acid salts, citric acid salts, and buffer solutions containing these salts. The buffers may be used singly, or two or more may be used in combination.

Examples of the soothing agents include, but are not limited to, benzyl alcohol.

Examples of the preservatives include, but are not limited to, sorbic acid, potassium sorbate, calcium sorbate, benzoic acid, sodium benzoate, propionic acid, sodium propionate, calcium propionate, sodium dehydroacetate, natamycin, pimaricin, polylysine, nisin, isopropyl paraoxybenzoate, isopropyl parahydroxybenzoate and isopropylparaben. The preservatives may be used singly, or two or more may be used in combination.

Examples of the antioxidants include, but are not limited to, sulfurous acid salts and ascorbic acid. The antioxidants may be used singly, or two or more may be used in combination.

Examples of the colorants include, but are not limited to, yellow ferric oxide, black iron oxide, edible yellow No. 4, edible red No. 3, tar dyes, caramel, titanium oxide and riboflavine. The colorants may be used singly, or two or more may be used in combination.

Examples of the sweeteners include, but are not limited to, sugars such as sucrose and fructose; sugar alcohols such as xylitol and sorbitol; artificial sweeteners such as aspartame, acesulfame potassium and sucralose; starch syrup, reduced starch syrup, high-fructose corn syrup and maple syrup. The sweeteners may be used singly, or two or more may be used in combination.

Examples of the surfactants include, but are not limited to, polysorbates, sodium laurylsulfate and polyoxyethylene hydrogenated castor oil. The surfactants may be used singly, or two or more may be used in combination.

Examples of the components useful for subjects include, but are not limited to, iron sources, trace metals, lipids, vitamins, nucleobases, nucleic acids, amino acids and derivatives thereof, proteins (including enzymes and the like), productive substances that produce bacteria, yeasts and the like, and other substances useful for treatment or health. These components may be used singly, or two or more may be used in combination.

Examples of the iron sources include, but are not limited to, transferrin, ferritin and iron (II) sulfate. The iron sources may be used singly, or two or more may be used in combination.

Examples of the trace metals include, but are not limited to, magnesium, zinc, cobalt, tin, molybdenum, nickel, selenium and related substances (including sodium selenite). The trace metals may be used singly, or two or more may be used in combination.

Examples of the lipids include, but are not limited to, docosahexaenoic acid and eicosapentaenoic acid. The lipids may be used singly, or two or more may be used in combination.

Examples of the vitamins include, but are not limited to, vitamin A (including vitamin A derivatives such as vitamin A acetates), vitamin B1, vitamin B2, vitamin B3 (niacin), vitamin B5 (pantothenic acid), vitamin B6, vitamin B12, vitamin C, vitamin D, vitamin E (including vitamin E derivatives such as DL-α-tocopherol acetate), vitamin K, biotin and folic acid. The vitamins may be used singly, or two or more may be used in combination.

Examples of the nucleobases include, but are not limited to, purine bases such as adenine and guanine; and pyrimidine bases such as thymine, cytosine and uracil.

The nucleobases may be used singly, or two or more may be used in combination.

Examples of the nucleic acids include, but are not limited to, DNA and RNA. The nucleic acids may be used singly, or two or more may be used in combination.

Examples of the amino acids and derivatives thereof include, but are not limited to, glycine, L-alanine, L-serine, L-valine, L-leucine, L-isoleucine, L-arginine, L-lysine, L-asparagine, L-glutamine, L-aspartic acid, L-glutamic acid, L-methionine, L-cysteine, L-proline, L-threonine, L-histidine, L-tryptophan, L-phenylalanine, L-tyrosine, L-carnitine, L-ornithine and glutathione (including the reduced form). The amino acids and derivatives thereof may be used singly, or two or more may be used in combination.

Examples of the proteins include, but are not limited to, animal proteins such as animal collagen, whey protein, casein and albumen; and vegetable proteins such as soybean protein and wheat protein. The proteins may be used singly, or two or more may be used in combination.

Examples of the productive substances that produce bacteria, yeasts and the like include, but are not limited to, yeast-producing substances and lactic acid bacteria-producing substances. The productive substances may be used singly, or two or more may be used in combination. Preferred productive substances are lactic acid bacteria-producing substances.

Examples of the components generally used in formulation and other processes include, but are not limited to, perfumes; flavors; fruit juices such as apple juice and mango juice; hard capsules, bases and the like which may be used in the production of hard capsule preparations; and bases, coating bases, plasticizers and the like which may be used in the production of soft capsule preparations. These components may be used singly, or two or more may be used in combination.

Examples of the hard capsules include, but are not limited to, carrageenan capsules and gelatinous capsules. The hard capsules may be used singly, or two or more may be used in combination.

Examples of the bases include, but are not limited to, aqueous bases such as water; and oil bases such as medium chain fatty acid triglycerides, tricaprylin, caproic acid, caprylic acid, oleic acid, linoleic acid, linolenic acid, coconut oil, olive oil, rapeseed oil, peanut oil, corn oil, soybean oil, cottonseed oil, grape oil and safflower oil.

The bases may be used singly, or two or more may be used in combination.

Examples of the coating bases include, but are not limited to, gelatin, succinylated gelatin, starch (including modified starch), pullulan, polyvinyl alcohol copolymer, macrogol, carrageenan and glycerin. The coating bases may be used singly, or two or more may be used in combination.

Examples of the plasticizers include, but are not limited to, sugar alcohols such as sorbitol and mannitol; and glycerin. The plasticizers may be used singly, or two or more may be used in combination.

In an embodiment of the present invention, the auxiliary component includes at least a component useful for a subject. In a preferred embodiment of the present invention, the auxiliary component includes at least one selected from the group consisting of nucleic acids, collagens, vitamin B1, vitamin B2, vitamin B6, vitamin B12, vitamin C, vitamin E, niacin, pantothenic acid, lactic acid bacteria-producing substances, docosahexaenoic acid and eicosapentaenoic acid.

The dose of the composition of the present invention may be about 8 to about 60 mg/kg body weight per day, preferably about 8 to about 40 mg/kg body weight per day, more preferably about 8 to about 30 mg/kg body weight per day, still more preferably about 8 to about 20 mg/kg body weight per day, and further preferably about 17 to about 20 mg/kg body weight per day, in terms of the compound having a structure of the formula I.

The composition of the present invention may be administered on any schedule without limitation as long as the above dose in terms of the compound having a structure of the formula I may be achieved. For example, the administration may be performed 1 to 4 times a day, preferably 1 to 3 times a day, more preferably 1 to 2 times a day, and still more preferably 2 times a day.

The composition of the present invention may be usually administered orally but may be administered parenterally (such as through injection, infusion or the like) as desired.

The ratio of the compound having a structure of the formula I to the auxiliary component is not particularly limited as long as the object of the present invention can be achieved. For example, the ratio may be 1 part by mass of the compound having a structure of the formula I to about 0.1 to about 100 parts by mass of the auxiliary component, preferably 1 part by mass of the compound having a structure of the formula I to about 1 to about 50 parts by mass of the auxiliary component, more preferably 1 part by mass of the compound having a structure of the formula I to about 1 to about 30 parts by mass of the auxiliary component, still more preferably 1 part by mass of the compound having a structure of the formula I to about 1 to about 16 parts by mass of the auxiliary component, and further preferably 1 part by mass of the compound having a structure of the formula I to about 7 to about 16 parts by mass of the auxiliary component.

Thus, an embodiment of the present invention resides in a composition described in the present specification which includes 1 to 16 parts by mass of the auxiliary component with respect to 1 part by mass of the compound having a structure of the formula I.

A preferred embodiment of the present invention resides in a composition described in the present specification which includes 7 to 16 parts by mass of the auxiliary component with respect to 1 part by mass of the compound having a structure of the formula I.

As used herein, the “subject having mild cognitive impairment” means a subject identified with brain wave analysis system NATESAS described later as having a similarity value with respect to general AD of more than 0.

Depending on symptoms such as the presence or absence of memory disorder, mild cognitive impairment may be classified into, for example, amnestic single domain MCI (the cognitive impairment that is present is memory disorder alone), amnestic multiple domain MCI (memory disorder is present among other cognitive impairments), non-amnestic single domain MCI (memory disorder is absent, and only one cognitive impairment other than memory is present), and non-amnestic multiple domain MCI (memory disorder is absent, and more than one cognitive impairments other than memory are present). The amnestic single domain MCI may progress to Alzheimer's dementia, etc. The amnestic multiple domain MCI may progress to Alzheimer's dementia, cerebrovascular dementia, etc. The non-amnestic single domain MCI may progress to frontotemporal dementia, etc. The non-amnestic multiple domain MCI may progress to Lewy body dementia, cerebrovascular dementia, etc.

For example, phrases such as “treating or preventing mild cognitive impairment” and “treatment or prevention of mild cognitive impairment” used herein mean that, although not limited to, (1) a healthy subject is prevented from developing mild cognitive impairment, (2) a subject having mild cognitive impairment is treated to or closer to a healthy state, and (3) the progression of mild cognitive impairment to dementia is delayed or prevented.

As used herein, the “brain wave analysis system” may be any system without limitation which can analyze brain waves of a subject and distinguish between a healthy person and a person with mild cognitive impairment. Examples of the brain wave analysis systems include, but are not limited to, the system described in Japanese Patent Application Kokai Publication No. 2016-106940. For example, brain wave analysis system NATESAS (registered trademark) (NIHON KOHDEN CORPORATION, NTT DATA i CORPORATION) (medical device certification number: 228AHBZX00042000) is preferable.

For example, Japanese Patent Application Kokai Publication No. 2016-106940 describes the following system.

A brain disease diagnosis assistance system includes: an evaluation model calculation unit which acquires a plurality of learning data items, each including brain wave feature data including feature amounts of brain waves extracted from the brain waves and disease information attached to the brain wave feature data, the disease information indicating a state of a brain disease corresponding to the brain wave feature data, and which calculates an evaluation model for performing brain disease determination, through machine learning of the plurality of learning data items; and a determination unit which acquires brain wave feature data of a subject and performs determination of a brain disease indicated by the brain wave feature data of the subject on the basis of the evaluation model. Of the plurality of feature amounts included in the brain wave feature data of each of the plurality of learning data items, the evaluation model calculation unit uses, in the calculation of the evaluation model, only feature amounts which are extracted through machine learning and are effective for the brain disease determination.

A brain disease diagnosis assistance apparatus is a system which presents a probability that a subject is suffering from dementia on the basis of brain waves of the subject. The brain disease diagnosis assistance apparatus is, for example, a PC or a server device which can execute a program realizing this functionality.

The brain disease diagnosis assistance apparatus includes a brain wave feature data acquisition unit, a manipulation-receiving unit, an evaluation model calculation unit, a determination unit, a storage unit, and a display unit.

The brain wave feature data acquisition unit can acquire brain wave feature data of a subject. The brain wave feature data is data including feature amounts of brain waves extracted from the brain waves of the subject. The brain waves may be, for example, data recording the changes in electrical potential difference between an electrode placed at an earlobe and another electrode from among electrodes placed at specific positions on the head of the subject according to the international 10-20 system. For example, the feature amounts of brain waves include squares (sNAT) of electrical potentials measured at the positions where the electrodes are placed, in each of a plurality of frequency bands, and ratios of sNAT values between adjacent frequency bands (which relate to the smoothness of the brain waves). The brain wave feature data acquisition unit can acquire learning data that include brain wave feature data and, attached thereto, disease information indicating a brain disease seen in individuals having the corresponding brain wave feature data.

The manipulation-receiving unit can receive an instructional manipulation that an operator has performed on the brain disease diagnosis assistance apparatus. Examples of the instructional manipulations include a manipulation instructing that brain wave feature data be loaded and a manipulation instructing that the brain wave feature data be evaluated.

The evaluation model calculation unit can calculate an evaluation model used for brain disease determination, through machine learning of a plurality of learning data items acquired by the brain wave feature data acquisition unit. For example, the evaluation model calculation unit calculates boundary information that separates the plurality of learning data items by the types of disease information attached to the respective learning data items. The machine learning used by the evaluation model calculation unit may be, for example, that of a support vector machine (SVM). The boundary information may be, for example, a function representing a separation interface which separates brain wave feature data of healthy persons from brain wave feature data of subjects with Alzheimer's dementia.

The determination unit can acquire the brain wave feature data of the subject and can perform determination of a brain disease indicated by the brain wave feature data of the subject on the basis of the evaluation model calculated by the evaluation model calculation unit. For example, the determination unit can evaluate brain wave feature data of a subject on the basis of the boundary information that the evaluation model calculation unit has calculated based on the learning data. The evaluation includes, for example, determining a brain disease corresponding to brain wave feature data of a subject from among a plurality of brain diseases, and calculating a probability that the brain disease determined from the brain wave feature data of the subject is correct.

The storage unit can store a variety of information such as brain wave feature data of a plurality of subjects and boundary information calculated by the evaluation model calculation unit.

The display unit can output an evaluation result from the determination unit to a display device connected to the brain disease diagnosis assistance apparatus.

Brain wave analysis system NATESAS is a system that can visualize brain waves from a subject by analyzing the brain waves using neuronal activity topography (NAT) technology. In the NAT technology, brain waves (waveforms) obtained are analyzed at frequencies, and the results are normalized in order to reduce the influence of factors such as differences among individuals, and are calculated as an arbitrary number of numerical markers (for example, about 420) which are then visualized (for example, into color maps or the like).

Brain waves that are analyzed with brain wave analysis system NATESAS may be usually brain wave data measured with an electroencephalograph (for example, one having 21 electrodes). When the brain waves thus obtained are analyzed with brain wave analysis system NATESAS, the intensities of the brain waves may be visualized (for example, as color maps) at frequencies (such as, for example, about 4.69 Hz or below, about 4.69 to about 6.25 Hz, about 6.25 to about 7.81 Hz, about 7.81 to about 9.38 Hz, about 9.38 to about 10.94 Hz, about 10.94 to about 12.50 Hz, about 12.50 to about 14.06 Hz, about 14.06 to about 15.63 Hz, about 15.63 to about 17.19 Hz, about 17.19 to about 18.75 Hz, and about 18.75 Hz or above, although not limited thereto). Further, brain wave analysis system NATESAS can compare statistically brain waves obtained from a subject to brain waves of healthy persons and/or subjects having a specific disease (such as, for example, Alzheimer's disease) constructed on brain wave analysis system NATESAS, thereby quantifying the brain waves into a similarity value.

As used herein, “general AD” refers to data that is obtained by comprehensive evaluation of brain waves categorized as CL1, CL2, CL3 and CL4 with brain wave analysis system NATESAS (for example, data obtained by grouping (clustering) similar brain waves into four feature patterns (CL1, CL2, CL3 and CL4) and averaging them in brain wave analysis system NATESAS).

As used herein, “CL1”, “CL2”, “CL3” and “CL4” are data obtained by categorizing brain waves of Alzheimer's disease patients into four types based on their similarities in brain wave analysis system NATESAS, and averaging the brain waves in the respective categories. For example, “CL2” may be classified as brain wave data of Lewy body dementia.

As used herein, the “similarity value” is a value obtained by analyzing brain wave data of a subject with NATESAS. The similarity value indicates the similarity between the brain waves of the subject and any of the brain waves of healthy persons, general AD, CL1, CL2, CL3 and CL4, the similarity being represented as a numerical value in the range of −1 to 1. Usually, a value of 0 or below means that the brain waves of the subject are similar to the brain waves of healthy persons, and a value of more than 0 means that the brain waves of the subject are similar to the brain waves of general AD, CL1, CL2, CL3 or CL4.

As used herein, the “membership probability” is a value obtained by analyzing brain wave data of a subject with NATESAS, and represents in % the similarity of the brain waves of the subject to CL1, CL2, CL3 or CL4. When, for example, the percentage value with respect to CL1 is highest, the brain waves of the subject may be judged as being most similar to the CL1 brain waves. To assess the therapeutic or preventive effects on mild cognitive impairment, changes in similarity to a category with a high percentage value, preferably the category with the highest percentage value, may be monitored over a predetermined period of time.

In an embodiment of the present invention, the subject identified as having mild cognitive impairment with a brain wave analysis system is a subject having a similarity value with respect to general AD of more than 0 as measured with brain wave analysis system NATESAS.

Examples of the subjects for the treatment or prevention of mild cognitive impairment include, but are not limited to, mammals including rodents such as mice, rats, hamsters and guinea pigs; lagomorphs such as rabbits; ungulates such as pigs, cows, goats, horses and sheep; carnivorans such as dogs and cats; and primates such as humans, monkeys, rhesus monkeys, cynomolgus monkeys, marmosets, orangutans and chimpanzees. Rodents and primates are preferable. Primates are more preferable, and humans are still more preferable.

For example, although not limited to, the compound having a structure of the formula I may be a commercially available compound or may be prepared by, for example, allowing an enzyme (such as, for example, phospholipase A1, phospholipase A2 or phospholipase B) to act on a compound having a structure of the formula 0:

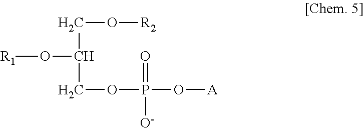

wherein R₁ and R₂ are each —COR₃,

R₃ independently at each occurrence is a saturated or unsaturated, C₁₋₃₀ aliphatic hydrocarbon group, and

A is —CH₂—CH₂—N⁺(CH₃)₃, —CH₂—CH₂—N⁺H₃, —CH₂—CH(NH₂)(COO⁻), or a C₃₋₆ cycloalkyl optionally substituted with 1 to 6 substituents selected from the group consisting of C₁₋₆ alkyls, C₁-6 alkoxies and hydroxyl groups. For example, the compound having a structure of the formula 0 may be a commercially available compound or may be a compound extracted from soybean, egg yolk or the like by a conventional method such as phospholipid extraction. The content of the compound with a structure of the formula I that is obtained as described above may be determined by a method such as, for example, a conventional phospholipid quantification method.

An example method will be described below in which a compound having a structure of the formula I is prepared from crude soybean oil.

Crude soybean oil is filtered, and water is added thereto at an elevated temperature of about 60° C. to about 80° C., the mixture being then stirred. The resultant precipitate composed of hydrous gum may be separated by centrifugation. Thereafter, the hydrous gum obtained is heated and dried under reduced pressure to give a paste composition, including a compound with a structure of the formula 0, that is composed of about 60 to about 65% by weight of phospholipids, about 35 to about 40% by weight of neutral fats, and trace amounts of free fatty acids and sterols. Next, water is added to the paste composition, and phospholipase A1 and/or A2 is allowed to act to hydrolyze predetermined fatty acid ester moieties of the phospholipids. After the completion of the reaction, the enzyme is inactivated by heating or the like. Further, drying is performed by heating under reduced pressure and is followed by filtration to give a mixture that includes a compound having a structure of the formula I.

Alternatively, thermally dried soybeans may be crushed and squeezed to give soybean oil, which is then decomposed using the enzyme described above, and treatments such as purification and filtration are performed as required to afford a mixture that includes a compound having a structure of the formula I.

The compound having a structure of the formula I may be a lysolecithin. In this case, the lysolecithin may be provided in any manner without limitation. For example, the lysolecithin may be purchased from the market or may be prepared by, for example, allowing an enzyme (such as, for example, phospholipase A1, phospholipase A2 or phospholipase B) to act on lecithin. For example, the lecithin used here may be one purchased from the market or may be one extracted from soybean, egg yolk or the like by a conventional method such as phospholipid extraction. The content of the lysolecithin that is obtained as described above may be determined by a method such as, for example, a conventional phospholipid quantification method.

The composition of the present invention may be formulated by methods which are known per se into preparations such as, for example, tablets, coated tablets, orally disintegrating tablets, chewable agents, pills, granules, fine granules, powders, hard capsule agents, soft capsule agents, liquid preparations (including, for example, syrups), suspensions, emulsions, jelly agents (including raw jelly agents) and inhalants.

For example, tablets may be prepared as follows.

Components such as the compound having a structure of the formula I, an excipient, a disintegrant and a binder are mixed together and granulated with water. The granules thus obtained are dried and are pulverized as required. Further, additional components such as a lubricant are added thereto, and the mixture is mixed and compacted into tablets.

For example, hard capsule agents may be prepared as follows.

Components such as the compound having a structure of the formula I and an excipient are mixed together, and additional components such as a lubricant are added thereto. The mixture is further mixed and is packed into hard capsules (such as, for example, gelatinous capsules), thereby forming hard capsule agents.

For example, soft capsule agents may be prepared as follows.

Components such as the compound having a structure of the formula I and an excipient are mixed together. The capsule contents thus obtained are packed into films using a known capsule filling device, thereby forming soft capsule agents.

For example, jelly agents may be prepared as follows.

Components such as the compound having a structure of the formula I, an excipient, a gelling agent and an emulsifier are mixed together. The mixture thus obtained is loaded into molds and is solidified by cooling, thereby forming a jelly agent.

For example, gummy agents may be prepared as follows.

Components such as an excipient and a gelling agent are mixed together, and additional components such as the compound having a structure of the formula I, and a gelling agent are added thereto, the mixture being mixed. Next, after further components such as an excipient are added as required, the mixture is poured into desired molds and is solidified by cooling or the like to form a gummy agent.

For example, liquid preparations may be prepared as follows.

Components such as the compound having a structure of the formula I, and a solvent are mixed together to form a liquid preparation.

The preparations formulated as described above may be used as, for example, therapeutic or prophylactic agents against mild cognitive impairment.

For example, the composition of the present invention or a preparation formulated with the composition may be used in foods or pharmaceuticals, and may be preferably used in foods.

For example, whether a person has mild cognitive impairment may be identified, and the therapeutic or prophylactic effects of the composition of the present invention against mild cognitive impairment may be assessed with use of brain wave analysis system NATESAS in the following manner Incidentally, brain wave analysis system NATESAS may be used with reference to the manual attached to the system.

An electroencephalograph may be attached to a subject to measure brain waves. The brain wave data obtained may be sent to brain wave analysis system NATESAS via a VPN line or the like and may be analyzed therein. The data analyzed by brain wave analysis system NATESAS may be received via a VPN line or the like. When the analysis shows a similarity value of more than 0 with respect to general AD, the subject may be identified as having mild cognitive impairment. Alternatively, color maps of the brain waves of the subject that are obtained by analysis with brain wave analysis system NATESAS may be compared to color maps of brain waves of healthy persons and/or patients such as Alzheimer's disease patients that are constructed on brain wave analysis system NATESAS to determine whether the subject has mild cognitive impairment.

A subject identified as having mild cognitive impairment may be made to ingest (preferably orally) the composition of the present invention on a schedule of, for example, 1 to 3 times a day, and brain waves may be measured similarly at time periods following the first ingestion, for example, after about 10 minutes, after about 20 minutes, after about 30 minutes, after about 40 minutes, after about 50 minutes, after about 1 hour, after about 2 hours, after about 4 hours, after about 6 hours, after about 12 hours, after about 1 day, after about 2 days, after about 3 days, after about 4 days, after about 5 days, after about 6 days, after about 1 week, after about 2 weeks, after about 1 month, after about 2 months, after about 3 months, after about 6 months, and after about 1 year, to determine similarity values with respect to CL1 to CL4 and general AD. If the similarity value with respect to general AD is more than 0 before the ingestion of the composition of the present invention and is lowered (preferably to 0 or below) by the ingestion of the composition of the present invention, the composition may be judged as being therapeutically or prophylactically effective against mild cognitive impairment.

Incidentally, brain wave analysis system NATESAS may be replaced by, for example, a similar brain wave analysis system that adopts the system described in Japanese Patent Application Kokai Publication No. 2016-106940, and whether a person has mild cognitive impairment may be identified, and the therapeutic or prophylactic effects of the composition of the present invention against mild cognitive impairment may be assessed in the manner described above or in a manner that is analogous thereto or that is appropriate for the assessment.

### EXAMPLES

The present invention will be described in more detail based on Examples described below. However, it should be construed that the scope of the present invention is not limited to such Examples.

### Example 1: Composition (i.e. Jelly Agent) Including Compound with Structure of Formula I, and Auxiliary Components

A stainless steel container was loaded with 63.9 parts by mass of water, and was further loaded with 16.7 parts by mass of excipients (including high-fructose corn syrup and maple syrup) and a gelling agent. The mixture was stirred at about 75 to about 90° C. to uniformity. There were added 0.5 parts by mass of an emulsifier and 0.06 parts by mass of soybean lysolecithin (a compound having a structure of the formula I), and the mixture was mixed. Thereafter, 14.94 parts by mass of fruit juice (including mango juice and apple juice), 0.25 parts by mass of citric acid, 1 part by mass of a perfume and 2.65 parts by mass of an excipient. The mixture was further stirred to give a composition (i.e. a jelly agent) of Example 1 (content of the compound having a structure of the formula I: 0.6 g/10 g of the jelly agent).

### Example 2: Composition (i.e. Gummy Agent) Including Compound with Structure of Formula I, and Auxiliary Components

A stainless steel container was loaded with 80 parts by mass of excipients (including starch syrup, reduced starch syrup and glucose). The excipients were heated to about 75 to about 90° C., and a solution of gelatin in a small amount of water at about 75 to about 90° C. was added thereto, the mixture being then stirred. There were added 0.072 parts by mass of an emulsifier and 9.375 parts by mass of soybean lysolecithin (a compound having a structure of the formula I), and the mixture was further mixed. Next, 0.641 parts by mass of fruit juice (including apple juice), 2.152 parts by mass of citric acid, 0.092 parts by mass of a perfume and 0.467 parts by mass of starch were added. The mixture was mixed to uniformity, poured into hemispherical molds to fill the same, and solidified by cooling to give a composition (a gummy agent) of Example 2 (3.2 g/grain, content of the compound having a structure of the formula I: 0.3 g/grain).

### Example 3: Composition (i.e. Soft Capsule Agent) Including Compound with Structure of Formula I, and Auxiliary Component

Capsule contents were obtained by mixing 47.2 parts by mass of soybean lysolecithin (a compound having a structure of the formula I) and 52.8 parts by mass of safflower oil. Next, the capsule contents obtained above were packed into films (including glycerin, gelatin and carrageenan) with use of an existing rotary die-type capsule forming apparatus. A composition (i.e. a soft capsule agent) of Example 3 was thus obtained (0.53 g/capsule, content of the compound having a structure of the formula I: 0.25 g/capsule).

### Example 4: Composition (i.e. Liquid Agent) Including Compound with Structure of Formula I, and Auxiliary Components

A composition (i.e. a liquid agent) of Example 4 was obtained by mixing 75.7 parts by mass of soybean lysolecithin (corresponding to a compound having a structure of the formula I), 20.5 parts by mass of safflower oil and 3.8 parts by mass of linseed oil (content of the compound having a structure of the formula I: 1.136 g/1.5 g of the liquid agent).

### Test Example 1: Evaluation 1 of Efficacy Against Mild Cognitive Impairment

The efficacy of the composition of the present invention against mild cognitive impairment was evaluated with respect to three subjects who had backgrounds described in Table 1. The three subjects had been all diagnosed as not having dementia by a medical interview.

TABLE 1

| | Sex | Age | Body weight |
|---|---|---|---|
| Subject 1 | Male | 61 years | 71 kg |
| Subject 2 | Female | 63 years | 59 kg |
| Subject 3 | Female | 67 years | 60 kg |

Brain waves of the subjects were measured (using 21 electrodes) with an electroencephalograph (manufactured by NIHON KOHDEN CORPORATION). The brain wave data obtained was analyzed with brain wave analysis system NATESAS (registered trademark) (medical device certification number: 228AHBZX00042000) (“before ingestion”). When the similarity to general AD that was obtained by this analysis was more than 0, the subject was identified as having mild cognitive impairment. As clear from the similarity values before ingestion described later in Tables 2 to 4, Subjects 1 to 3 were identified as having mild cognitive impairment.

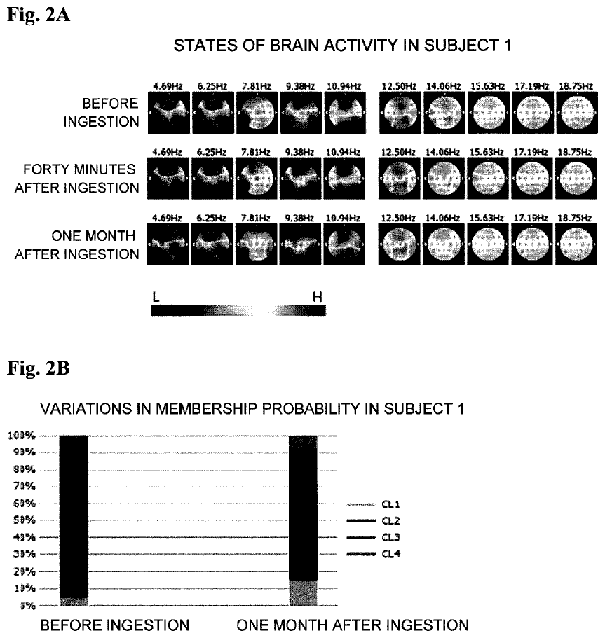

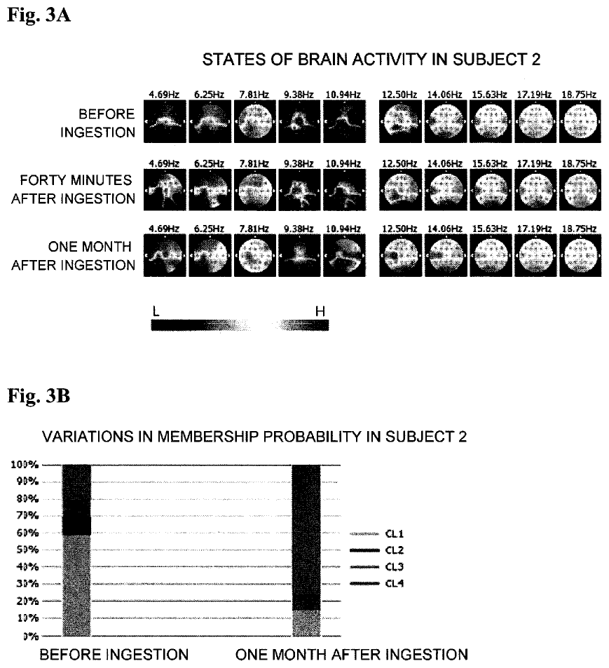

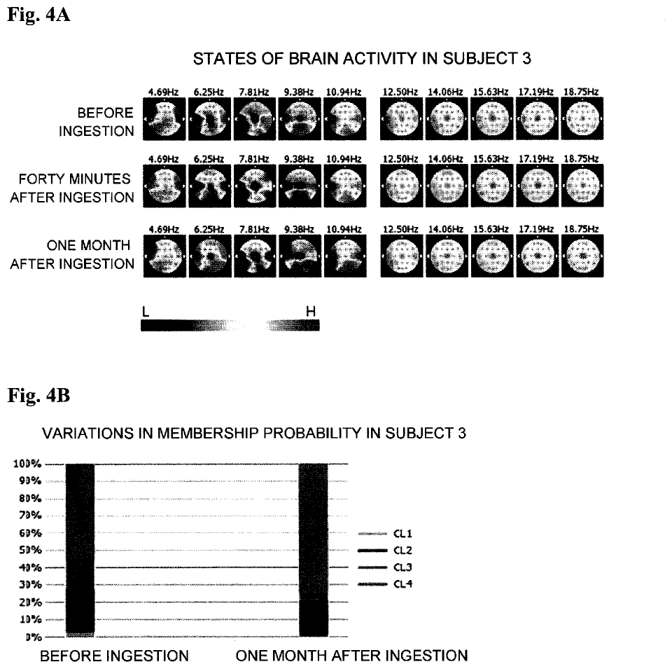

Next, Subjects 1 to 3 identified as having mild cognitive impairment received oral ingestion of 2 packages of the composition of Example 1 (18.8 g, 1.2 g in terms of the compound having a structure of the formula I; about 17 to about 20 mg of the compound having a structure of the formula I per kg body weight). Forty minutes after the ingestion, brain waves were measured with the electroencephalograph, and the brain wave data obtained was analyzed with brain wave analysis system NATESAS (“forty minutes after ingestion”). Further, Subjects 1 to 3 orally ingested the composition of Example 1 continuously for about 1 month from the next day after being identified as having mild cognitive impairment, 3 times a day (morning, afternoon and night), 1 to 2 packages at a time, with a total of 4 packages per day (37.6 g, 2.4 g in terms of the compound having a structure of the formula I; about 34 to about 41 mg of the compound having a structure of the formula I per kg body weight). Brain waves were measured again (“one month after ingestion”). The results are shown in Tables 2 to 4 and FIGS. 2A to 4B.

TABLE 2
Changes in similarity values in Subject 1

| | Before | Forty minutes | One month after |
|---|---|---|---|
| Similarities | ingestion | after ingestion | ingestion |
| General AD | 0.36 | −0.16 | −0.12 |
| CL1 | −0.67 | −0.74 | −0.57 |
| CL2 | 0.88 | 0.84 | 0.56 |
| CL3 | −0.96 | −0.97 | −0.94 |
| CL4 | −0.96 | −0.93 | −0.84 |

TABLE 3
Changes in similarity values in Subject 2

| | Before | Forty minutes | One month after |
|---|---|---|---|
| Similarities | ingestion | after ingestion | ingestion |
| General AD | 0.55 | −0.42 | −0.43 |
| CL1 | 0.79 | 0.63 | −0.13 |
| CL2 | −0.99 | −0.90 | −0.97 |
| CL3 | −0.94 | −0.98 | −0.94 |
| CL4 | 0.41 | −0.95 | −0.69 |

TABLE 4
Changes in similarity values in Subject 3

| | Before | Forty minutes | One month after |
|---|---|---|---|
| Similarities | ingestion | after ingestion | ingestion |
| General AD | 0.94 | 0.89 | 0.87 |
| CL1 | −0.83 | −0.86 | −0.89 |
| CL2 | 0.95 | 0.75 | 0.83 |
| CL3 | 0.33 | 0.28 | −0.41 |
| CL4 | 0.94 | 0.94 | 0.96 |

In Subject 1, as clear from Table 2, the similarity value with respect to general AD was more than 0 before oral ingestion, but the value decreased to below 0 in 40 minutes after oral ingestion of the composition of the present invention. After about 1 month of continuous oral ingestion of the composition of the present invention, the similarity value with respect to general AD was below 0 and the similarity value with respect to CL2 decreased.

In Subject 2, as clear from Table 3, the similarity values with respect to general AD and CL4 were more than 0 before oral ingestion, but the values decreased to below 0 in 40 minutes after oral ingestion of the composition of the present invention. Further, the similarity value with respect to CL1 was more than 0 before oral ingestion and 40 minutes after oral ingestion, but the value decreased to below 0 after about 1 month of continuous oral ingestion of the composition of the present invention.

In Subject 3, as clear from Table 4, the similarity value with respect to CL3 was more than 0 before oral ingestion and 40 minutes after oral ingestion, but the value decreased to below 0 after about 1 month of continuous oral ingestion of the composition of the present invention. Further, the similarity value with respect to general AD decreased 40 minutes after oral ingestion and 1 month after oral ingestion as compared to the value before oral ingestion.

As shown by the results of Examples described above, the compositions provided according to the present invention can treat or prevent mild cognitive impairment. Further, as clear from the results of Examples, the compositions of the present invention can bring brain waves of a subject having mild cognitive impairment to or close to brain waves of a healthy person, and thus allow for the radical treatment of dementia and the radical prevention of the onset of dementia which are far from possible with the conventional therapeutic agents, methods and other approaches.

What is claimed is:

1. A method for treating or preventing mild cognitive impairment in a subject identified as having mild cognitive impairment with a brain wave analysis system, the method comprising:
a step of bringing brain waves of the subject to or close to brain waves of a healthy person by administering to the subject a composition comprising a compound having a structure represented by the formula I below and an auxiliary component, 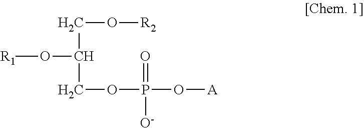
wherein R₁ and R₂ are each independently hydrogen or —COR₃ with the proviso that R₁ and R₂ are not —COR₃ at the same time,
R₃ is a saturated or unsaturated, C₁₋₃₀ aliphatic hydrocarbon group, and
A is —CH₂—CH₂—N⁺(CH₃)₃, —CH₂—CH₂—N⁺H₃, —CH₂—CH(NH₂)(COO⁻), or a C₃₋₆ cycloalkyl optionally substituted with 1 to 6 substituents selected from the group consisting of C₁₋₆ alkyls, C₁₋₆ alkoxies and hydroxyl groups,
the dose of the composition administered to the subject in the step being 8 to 60 mg/kg body weight per day in terms of the compound having a structure of the formula I.

2. The method according to claim 1, wherein in the compound having a structure of the formula I, R₁ is hydrogen and R₂ is —COR₃.

3. The method according to claim 1, wherein in the compound having a structure of the formula I, A is —CH₂—CH₂—N⁺(CH₃)₃.

4. The method according to claim 1, wherein the compound having a structure of the formula I is a lysolecithin.

5. The method according to claim 4, wherein the lysolecithin is soybean lysolecithin.

6. The method according to claim 1, wherein the subject identified as having mild cognitive impairment with a brain wave analysis system is a subject having a similarity value with respect to general AD of more than 0 as measured with brain wave analysis system NATESAS.

7. The method according to claim 1, wherein the composition includes the auxiliary component in an amount of 1 to 16 parts by mass per part by mass of the compound having a structure of the formula I.

8. The method according to claim 1, wherein the auxiliary component comprises at least one selected from the group consisting of nucleic acids, collagens, vitamin B1, vitamin B2, vitamin B6, vitamin B12, vitamin C, vitamin E, niacin, pantothenic acid, lactic acid bacteria-producing substances, docosahexaenoic acid and eicosapentaenoic acid.
